(19) United States
(12) Patent Application Publication
(10) Pub. No.: US 2015/0031997 A1
(43) Pub. Date: Jan. 29, 2015

(54) TECHNIQUE FOR THE DELIVERY OF ELECTROMAGNETIC ENERGY TO NANOPARTICLES EMPLOYED IN MEDIAL TREATMENT

(71) Applicant: INTEGRAL ELECTROMAGNETRONIC TECHNOLOGIES LLC, Dover, DE (US)

(72) Inventors: Albert Francis Messano, Montavale, NJ (US); Mandeep Singh, Lodi, NJ (US)

(73) Assignee: INTEGRAL ELECTROMAGNETRONIC TECHNOLOGIES LLC, Dover, DE (US)

(21) Appl. No.: 14/515,126

(22) Filed: Oct. 15, 2014

Related U.S. Application Data
(63) Continuation of application No. 13/564,054, filed on Aug. 1, 2012, now abandoned.
(60) Provisional application No. 61/573,017, filed on Aug. 5, 2011.

(51) Int. Cl. A61B 5/05; A61B 5/00

(52) U.S. Cl. CPC A61B 5/05; A61B 5/0022; A61B 5/742
USPC 600/431

(57) ABSTRACT

The present invention relates to technology for delivering electromagnetic (EM) energy to nanoparticles (nanos) utilized in the treatment of either existing or potential medical conditions. Nanotechnology is increasingly being used to deliver various types of treatments and remedies for existing medical conditions. Potentially, nanotechnology may be used in an inoculation mode to protect a patient from incurring future medical conditions. Such treatments, either real-time or proactive, may require a method of energizing nanoparticles or nanodevices (collectively referred to as nanos) energized in a noninvasive manner. Similarly nanodoctors or nanosurgeons operating in situ (within the human body) may require a method of being energized.

### CROSS-REFERENCE TO RELATED APPLICATIONS

This application is a continuation of U.S. patent application Ser. No. 13/564,054 filed Aug. 1, 2012, which claims the priority of U.S. Patent Application No. 61/573,017 filed Aug. 5, 2011. The entire disclosures of U.S. patent application Ser. Nos. 13/564,054 and 61/573,017 are expressly incorporated herein by reference thereto.

### TECHNICAL FIELD

This invention relates to technology for delivering electromagnetic (EM) energy to nanoparticles (nanos) utilized in the treatment of either existing or potential medical conditions.

### BACKGROUND

Nanotechnology is increasingly being used to deliver various types of treatments and remedies for existing medical conditions. Potentially, nanotechnology may be used in an inoculation mode to protect a patient from incurring future medical conditions. Such treatments, either real-time or proactive, may require a method of energizing nanoparticles or nanodevices (collectively referred to as nanos) energized in a noninvasive manner. Similarly, nanodoctors or nanosurgeons operating in situ (within the human body) may require a method of being energized and/or directed to the site of treatment. Thus, it would be beneficial to provide a safe and non-invasive biomedical energy delivery and orienting technique that obviates at least some of the limitations of existing technology.

### SUMMARY OF THE INVENTION

This invention relates to technology for delivering electromagnetic (EM) energy to nanoparticles (nanos) utilized in the treatment of either existing or potential medical conditions and/or for directing nanos to the site of treatment.

The general technique utilized is to expose a portion of the nanos utilized in the treatment of the test subject or patient to low doses of radio frequency (RF) electromagnetic energy. Different biomaterials in a nanoparticle may be differentiated and identified by characterizing their electromagnetic properties based on observed parameters (e.g. electromagnetic energy absorbed, thermal energy created, and electromagnetic energy emitted), during irradiation of the nanos utilized in the treatment of the test subject or patient.

In accordance with one aspect of the invention, provided is a system for multispectral scanning and detecting biomaterials in nanos utilized in the treatment of the test subject or patient. In one embodiment, the system may comprise a scanning module and a detection module. The scanning module is preferably adapted to deliver electromagnetic energy to the nanos by radiation at selected frequencies and power. The detection module is preferably adapted to detect RF electromagnetic radiation reflected by nanos and infrared (IR) electromagnetic radiation emitted by the nanos for the purpose of evaluating the RF dosage applied to the patient.

In another embodiment, the system may further comprise a processing module, a control module, and a data module. The processing module is preferably connected to the scanning and detecting modules so that it can perform calculations for the control and data modules. The control module is preferably connected to the scanning module, detection module, and processing module in order to control the timing, power level, antenna gain, and scan frequency of the scanning module. The data module preferably processes data from the processing module and an optional imaging module, and structures the data into video format.

In accordance with another aspect of the invention, provided are methods for multispectral scanning and detection of biomaterials in a nanos utilized in the treatment of the test subject or patient. In one implementation, a method for multispectral scanning and detection of biomaterials comprises irradiating at least a portion up to all of the nanos with RF electromagnetic radiation, detecting IR electromagnetic radiation emitted by the irradiated nanos, and providing data from the irradiated nanos to evaluate the dosage of biomaterials.

In another implementation, the method of scanning and detection may further comprise measuring and/or calculating parameters of the RF electromagnetic radiation impinged on the nanos and adjusting irradiation of the nanos to comply with Federal Communications Commission (FCC) Maximum Permitted Exposure (MPE) limits while maximizing the depth of penetration to ensure proper scanning of the nanos.

In another implementation, the method of scanning and detection may further comprise measuring and/or calculating parameters of the nanos utilized in the treatment of the test subject or patient during irradiation; calculating electromagnetic properties of biomaterials in the nanos utilized in the treatment of the test subject or patient based on the measured and/or calculated parameters of the nanos utilized in the treatment of the test subject or patient during irradiation; and differentiating and/or identifying biomaterials in the nanos utilized in the treatment of the test subject or patient based on the electromagnetic properties of different biomaterials.

These and other aspects of the invention will become apparent from the present specification and claims.

### BRIEF DESCRIPTION OF THE DRAWING FIGURES

FIG. 1 is a flowchart of an exemplary system for delivery of electromagnetic energy to nanoparticles and

FIG. 2 is a schematic illustration of an exemplary implementation of a method for delivery of electromagnetic energy to nanoparticles.

### DETAILED DESCRIPTION OF THE INVENTION

Reference will now be made in detail to the embodiments of the invention, examples of which are illustrated in the accompanying drawings. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention as defined by the appended claims. Furthermore, in the following detailed description of the present invention, numerous specific details are set forth in order to provide a thorough understanding of the present invention. It will be obvious to one of ordinary skill in the art that the present invention may also be practiced without all specific details. In other instances, well-known methods, procedures, components, and circuits have not been described in detail so as not to unnecessarily obscure aspects of the present invention.

This invention relates to the technology for the delivery of electromagnetic energy to nanoparticles utilized in the treatment of the test subject or patient for treatment of or for diagnosing existing and potential medical conditions. The general technique utilized is to expose a portion of the nanos to low doses of RF electromagnetic energy. Some of the RF electromagnetic energy radiated to the nanos is absorbed by the nanos and converted into thermal energy. The nanos is comprised of different biomaterials having different electromagnetic properties, and therefore, electromagnetic energy is absorbed differentially by the nanos. As a result, different nanos in the nanos utilized in the treatment of the test subject or patient absorb RF energy at different rates.

The electromagnetic properties of the nanos determine how much RF electromagnetic energy is absorbed, converted into thermal energy, and emitted as IR electromagnetic energy. Thus, different nanos may be differentiated and identified by characterizing their electromagnetic properties based on observed parameters of the biomaterials (e.g. electromagnetic energy absorbed, thermal energy created, and electromagnetic energy emitted).

### System

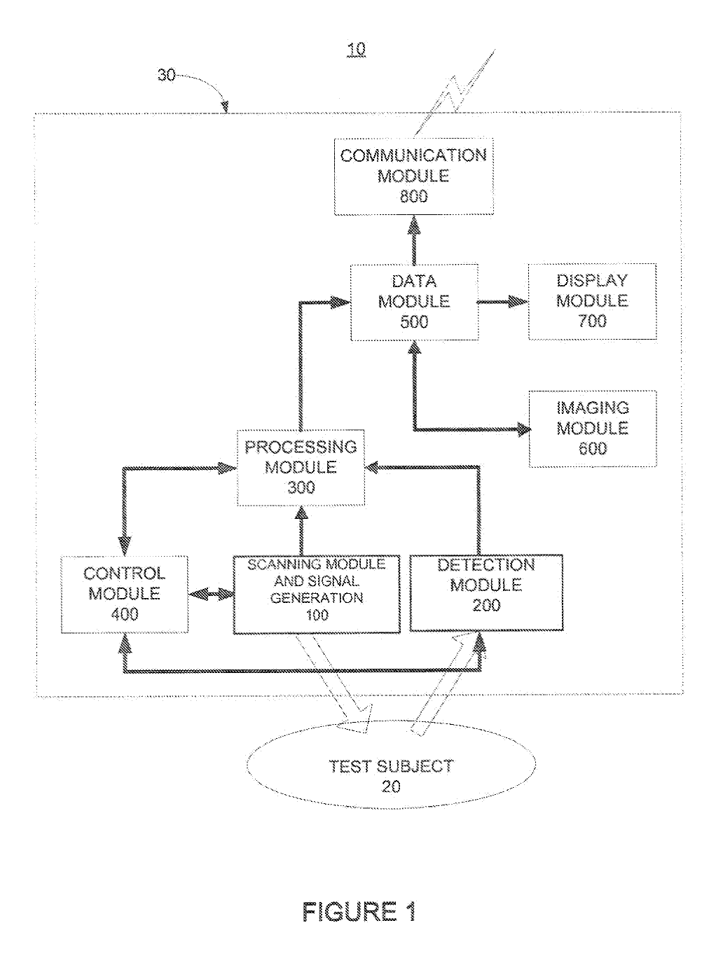

In accordance with one aspect of the invention, provided is a system 10 for scanning and detecting biomaterials in nanos utilized in the treatment of the test subject or patient 20. In one embodiment as shown in FIG. 1, system 10 may comprise a scanning module 100, a detection module 200, a processing module 300, a control module 400, a data module 500 and a communication module 800. In the embodiment shown, system 10 is organized into separate modules, but one skilled in the art will appreciate that one of these modules or portions thereof may be combined with another of these modules or portions thereof. The various modules of system 10 are described in further detail below. The headings provided herein are for convenience only and do not interpret the scope or meaning of the claimed invention.

Preferably, system 10 may be contained in a portable and robust package 30 with a footprint the size of a small LCD display (approximately 18″ by 12″) so that system 10 may be easily deployed in field applications. Further, system 10 is preferably packaged in a unit of roughly the same weight as a standard laptop and is preferably powered by a standard Lithium-ion battery, such as those used for laptop computer applications.

### Scanning Module

Scanning module 100 is adapted to deliver electromagnetic energy to nanos utilized in the treatment of the test subject or patient 20 by radiation at selected frequencies and power. Preferably, the electromagnetic energy that is radiated to nanos utilized in the treatment of the test subject or patient 20 is in the radio frequency range of the electromagnetic spectrum.

In one embodiment, scanning module 100 comprises a signal generator coupled to an antenna that amplifies and transmits electromagnetic energy to nanos over an RF path. As such, scanning module 100 is subject to FCC regulation. The FCC establishes guidelines for operations and devices to comply with limits for human exposure to RF fields adopted by the FCC and publishes these guidelines in OET Bulletin 65. According to FCC guidelines, the limits of MPE to electromagnetic radiation with frequencies of 0.3 MHz-3.0 MHz are Power Density (S) of 100 mW/cm2, Electric Field Strength (E) of 614 V/m, and magnetic field strength of (H) of 1.6 A/m.

The antenna incorporated in the Scanning Module exposes the nanos in or on the patient or test subject to low doses of non-ionizing RF electromagnetic radiation. Preferably, the signal generator has a variable power output of 1 mW-100 mW and a duty cycle of 50% or less. Further, the signal generator is preferably adapted to produce non-ionizing electromagnetic radiation having a frequency of 1 GHz-40 GHz. The antenna is designed to bathe nanos with RF electromagnetic radiation from the signal generator. Preferably, the antenna is adapted to amplify the signal power of the electromagnetic radiation produced by the signal source with a variable Gain (G) of 1 dB-10 dB. Thus, the net amplified power output (Rₜ) from scanning module 100 depends on the output power (Pₜ) of the signal generator as well as the gain (G) of the antenna. This net amplified power output (Rₜ) may be described many ways, including Effective Isotropic Irradiated Power (EIRP) and Effective Radiated Power (ERP). For purposes of this discussion, net amplified power output (Rₜ) will be referred to as the EIRP, which is calculated as follows:

EIRP=G*Pₜ

As shown by the above equation, the EIRP of scanning module 100 may be controlled by adjusting the gain of the antenna and/or the power output of the signal generator.

Further, in another embodiment, scanning module 100 may also comprise a sonic proximity detector that is adapted to sense the length of the RF path (e.g., the distance that the electromagnetic radiation travels from the antenna to nanos).

The electromagnetic radiation delivered by scanning module 100 is subject to attenuation or energy loss due to the distance traveled from the antenna to nanos. This attenuation is a function of the distance from the antenna to nanos, the material between the antenna and the nanos (e.g. air), and the frequency of the electromagnetic radiation. Thus, in order to deliver the maximum allowable electromagnetic radiation while complying with FCC limits of Maximum Permitted Exposure (MPE), the attenuation of electromagnetic radiation with frequencies of 0.3 MHz-3.0 MHz must be monitored to ensure that the Power Density (S) is below 100 mW/cm2 or the applicable FCC MPE limit. The amount of power transmitted to nanos will vary as control module 400 adjusts scanning module's 100 output power Pₜ, antenna Gain G, and frequency to obtain maximum penetration of nanos utilized in the treatment of the test subject or patient 20 while complying with MPE limits.

### Detection Module

Detection module 200 is adapted to detect RF electromagnetic radiation reflected by nanos and IR electromagnetic radiation emitted by nanos as absorbed RF electromagnetic radiation from the signal generator is converted into thermal energy by nanos. Detection module 200 scans nanos being irradiated with RF electromagnetic energy and can detect the amount of RF electromagnetic energy reflected and the amount of IR electromagnetic energy emitted by nanos. When irradiated with RF electromagnetic energy of a given frequency, different biomaterials absorb and convert RF electromagnetic energy into thermal energy at different rates, and as a result, emit IR electromagnetic energy at different rates. Thus, by examining the RF electromagnetic energy reflected and the IR electromagnetic energy emitted by different portions of nanos, different biomaterials in a nanoparticle may be differentiated and identified. The data collected by detection module 200 may be processed, conditioned, and formatted by an optional imaging module 600 to make a thermal image of nanos available to both a local operator through a display module 700 and to a remote operator through communication module 800. Further, the data collected by detection module 200 may be communicated to processing module 300 and data module 500 for differentiating and identifying the biomaterials of nanos Additionally, the display module 700 may provide local medical personnel, such as EMT's, with exposure, safety and system information. An optional imaging module 600 may be provided for real-time diagnostics of the patient or test subject by medical professional on location.

Detection module 200 comprises an IR camera and a detector. In one embodiment, the IR camera and detector preferably comprise a charge-coupled device (CCD) that senses IR electromagnetic radiation and produces analog electrical signals that are converted to digital signals for display as an image. Preferably, the CCD has a range of 4.0 μm to 21.0 μm wavelengths, which are considered Mid-Wave IR (MWIR) to Long-Wave IR (LWIR). The purpose of such monitoring is to ensure that the proper dose of RF energy is being applied at the correct therapeutic site in compliance with FCC MPE limits.

Detection module 200 preferably is sensitive to differential thermal heating of nanos of 2° F. to 5° F. and differential thermal emission of nanos of up to about 3.0 mW/cm². Further, the CCD preferably has a nominal sensitivity of 0.1° K, a resolution of approximately 2048×2048 pixels, and a data rate of between 20 MHz and 50 MHz. Also, the CCD is preferably capable of 16-bit analog-to-digital signal conversion. The IR return path from a Beam Steering Ultrasonic Transducer (BSUT) to the Detector Module 200 does not critically affect the data transfer.

### Processing Module

Processing module 300 performs calculations that may be required by control module 400 and data module 500. Preferably, processing module 300 comprises either a microprocessor (μP) or an application-specific integrated circuit (ASIC) configured to receive input signals from scanning module 100 and/or detection module 200, perform calculations, and transmit output signals to control module 400 and/or data module 500. Thus, in one embodiment, processing module 300 may be connected to scanning, and detecting modules so that it can receive operational data, perform calculations, and communicate signals/data to control module 400 and data module 500.

In accordance with one embodiment processing module, processing module 300 receives operational data from scanning module 100 and performs calculations to determine different aspects of the system's 10 performance, such as EIRP, power density (S), power received (Pᵣ), path loss, power incident, and power reflected. Information may stream from the control module 400, scanning module 100 and detection module 200 to be received by the processing module 300. Therefore, the processing module 300 may provide an overall level of system oversight for safety and operational purposes. The processing module 300 may further prepare the control and data stream for the data module 500. The data module 500 may then preprocess the information stream and transmit the information stream to the communication module 800 and/or the display module 700.

For example, processing module 300 may perform calculations to determine whether system 10 is operating within the FCC's MPE limits for power density. By receiving operational data from scanning module 100 regarding the gain, output power, and distance to nanos, processing module 300 may calculate the power density of the electromagnetic energy delivered to the nanos and may send a corresponding signal to control module 400 which can adjust operation of scanning module 100 to comply with the MPE limits for power density per the FCC guidelines.

Thus, process module 300 may be programmed to perform at least the calculations explained in detail below. For example, the net amplified power output (e.g., the Effective Isotropic Irradiated Power (EIRP)) of scanning module 100 may be calculated using the equation:

EIRP=G*Pₜ,

where G is the antenna gain and Pₜ is the power output of the signal generator. The antenna gain G and the power output Pₜ are transmitted by scanning module 100 to process module 300. Further, the power density (S), as defined by FCC OET 65, may be calculated using the equation:

S=EIRP/4πR²,

where EIRP is the effective isotropic irradiated power of scanning module 100 and R is the distance between the antenna and the nanos. The distance R is determined by scanning module's 100 proximity detector and transmitted to processing module 300. Thus, by receiving operational data from scanning module 100 regarding the gain G, power output Pₜ, and distance R, processing module 300 can determine whether scanning module 100 is operating within the FCC MPE limits for power density.

Processing module 300 will perform these calculations and provide control module 400 with a signal output corresponding to the power density (S) of system 10. Thus, control module 400 can compare the signal output corresponding to the power density (S) of system 10 to a reference value corresponding to the FCC MPE limit for power density and adjust operation of scanning module 100 accordingly. Again, both the gain G and the power output Pt may be adjusted by control module 400 to maintain the prescribed FCC MPE limit at the outer surface of the nanos while maximizing power output and penetration depth.

Also, processing module 300 may be adapted to perform calculations to determine the actual power delivered to nanos, accounting for attenuation or power loss of the electromagnetic radiation as it travels from the antenna to nanos. For example, the actual power received Pᵣ by nanos may be determined by using a variant of the well-known Friis Equation:

Pᵣ=PₜGₜGᵣ(λ/4πR)²,

where Pᵣ is the power received by nanos, Pₜ is the power transmitted by the signal generator, Gₜ is the antenna gain, Gᵣ is the gain of nanos (assumed to have no gain, i.e. equal to 1), λ is the wavelength of the electromagnetic energy transmitted, and R is the distance between the antenna and nanos. Scanning module 100 communicates operational data, such as gain Gₜ, power transmitted Pₜ, and wavelength λ, to processing module 300. Scanning module's 100 proximity detector determines the distance R and communicates it to processing module 300. Thus, by receiving operational data from scanning module 100 regarding the gain G, power output Pₜ, wavelength λ, and distance R, processing module 300 can determine the actual power received Pᵣ by nanos. By calculating the difference between the power delivered EIRP to nanos and the actual power received Pᵣ by the nanos, the energy loss along the length of the RF path (i.e. path loss) may be determined.

In accordance with another aspect of the present invention, processing module 300 may perform calculations to approximate certain electromagnetic properties of the biomaterials based on various nanos parameters measured and calculated by processing module 300. Thus, processing module 300 may calculate electromagnetic properties of different biomaterials so that the biomaterials may be differentiated and identified.

For example, the index of refraction (n) of a biomaterial may be calculated using the well-known Frenel Equations:

Tₙ=1−Rₙ, and

Rₙ=Rₛ=Rₚ=((n₁−n₂)/(n₁+n₂))²,

where Tₙ is the incident power, Rₙ (R, Rₛ or Rₚ) is the reflected power, and n is the index of refraction of the biomaterial. The subscripted symbols refer to either the transverse or parallel components of the Transmitted Power, Tₙ, and the reflected Power, Rₙ. The index of refraction, n, is an electromagnetic property of all materials, even biomaterials. The incident power or transmitted power Tₙ may also be referred to as the incident power Pᵢ.

Further, based on measured and calculated parameters of the nanos (such as attenuation α of electromagnetic radiation, absorption/reflection of electromagnetic radiation, depth of penetration electromagnetic radiation, emission of IR electromagnetic radiation, and change in temperature), various electromagnetic properties of the biomaterials in the nanos may be calculated, such as relative static permittivity (ε), magnetic permeability (μ), and thermal conductivity (κ).

For example, the thermal conductivity (κ) of a biomaterial may be calculated by solving the equation:

T_{f}=Tᵢ+Q/κ,

where T_{f} is the final temperature of the biomaterial, Tᵢ is the initial temperature of the biomaterial, and Q is the amount of energy added to the biomaterial (or the transmitted power Tₙ as described above). The initial temperature Tᵢ and the final temperature T_{f} may be measured by detection module 200. The amount of energy added (Q) to the biomaterial may be calculated by processing module 300 based on measurements from scanning module 100 as explained above.

Once the electromagnetic properties of the nanos are established, the nanoparticles may be differentiated and/or identified by data module 500 to detect any anomalies.

### Control Module

In one embodiment, control module 400 is connected to at least scanning module 100, detection module 200, and processing module 300. Preferably, control module 400 is connected to other modules via USB, BioBus, or other communication protocol that allows communication of signals/data among the modules. Control module 400 is adapted to control the timing, power level, antenna gain, and scan frequency of the signal generator of scanning module 100. The generator's frequency is preferably variable between 1.0 GHz and 40.0 GHz, considered to be in the radio-frequency range. Further, control module 400 is adapted to control the detection wavelengths of detection module 200. Preferably, detection module 200 operates in a wavelength range of 4.0 μm to 21.0 μm, considered mid-wave infrared (MWIR) to long-wave infrared (LWIR).

One of the primary functions of control module 400 is to ensure that operation of scanning module 100 is within the MPE limits set forth by FCC guidelines, which currently set a maximum power density of 100 mW/cm². Additionally, control module 400 is preferably adapted to adjust the power density (S) and frequency of the electromagnetic energy delivered in order to maximize the depth of penetration and ensure proper scanning of the nanos. In order to optimize scanning of nanos while still complying with FCC MPE limits, control module 400 is arranged in a control feedback loop that allows it to monitor and adjust operation of scanning module 100. The control module 400 may also blend data from both the scanning module 100 and detection module 200 with operational data generated by the control module 400 in order to perform calculations to determine exposure, safety and other system parameters. Accordingly, both the exposure and sensitivity may be adjusted by the control module 400 to meet dosage requirements and operational parameters.

As shown in FIG. 1, control module 400 is connected to scanning module 100. Thus, control module 400 controls the gain of the antenna and the power output of the signal generator to produce a power density (S) of less than 100 mW/cm² or the applicable FCC limit. Further, control module 400 is connected to scanning module 100 via processing module 300. Processing module 300 may perform calculations to determine whether system 10 is operating within the FCC's MPE limits for power density. For example, by receiving operational data from scanning module 100 regarding the gain, output power, and distance to the nanos, processing module 300 may calculate the power density of the electromagnetic energy delivered to the nanos and may send a corresponding signal to control module 400. Thus, control module 400 is adapted to compare the signal output corresponding to the power density (S) of system 10 to a reference value corresponding to the FCC MPE limit for power density and adjust operation of scanning module 100 accordingly. It should be pointed out that should the FCC guidelines regarding the MPE limits be updated or replaced, control module 400 may be reprogrammed to ensure compliance.

Additionally, control module 400 is connected to detection module 200 via processing module 300. Processing module 300 may calculate the depth of penetration of the electromagnetic energy delivered to the nanos based on the nanoparticle parameters measured by detection module 200. Thus, control module 400 may communicate with processing module 300 to determine whether the power output, antenna gain, and/or frequency of scanning module 100 may be adjusted to increase the depth of penetration of the electromagnetic energy delivered to nanos while still complying with the FCC MPE limits.

### Data Module

In one embodiment, data module 500 may be connected to processing module 300, an optional imaging module 600, a display module 700, and a communication module 800. Data module 500 processes data from processing module 300 and the optional imaging module 600 and structures the data into video format for representation in the display module 700 and also prepares the data for wireless transmission via communication module 800. Preferably, data module 500 is connected to other modules via USB, BioBus, or other communication protocol that allows communication of signals/data among the modules.

In accordance with one embodiment, data module 500 receives IR radiation emission data corresponding to different locations on nanos in response to irradiation at a given frequency and compares the data to known measurements of IR radiation emission for various biomaterials in response to irradiation at the same frequency. Data regarding how much IR radiation different biomaterials emit after being irradiated with RF radiation of a particular frequency or wavelength may be stored and accessed in one or more lookup tables in data module 500. Thus, data module 500 may identify and/or differentiate biomaterials based on the frequency/wavelength of the IR radiation emitted in response to irradiation with RF radiation of a given frequency. Additionally, data module 500 may receive data regarding electromagnetic properties of biomaterials in different locations in nanos from processing module 300. Data regarding various electromagnetic properties of different biomaterials may be stored and accessed in one or more lookup tables in data module 500 so that data module 500 may identify and/or differentiate different biomaterials in nanos. Based on the data received from the optional imaging module 600 and processing module 300, data module 500 may differentiate and/or identify the biomaterials comprising nanos by providing a graphical representation of the different biomaterials via a display module 700. Particularly, data module 500 may differentiate diseased or precursor tissue from normal tissue, and thus allow detection of anomalies.

### Communication Module

In one embodiment, communication module 800 is connected to data module 500 and is preferably configured to have wireless access to both local and wide-area networks (LAN's and WAN's) using existing communication protocols, such as Bluetooth, WiFi, WiMax or the like. Communication module 800 is adapted to allow sharing of diagnostic information with medical professionals and accessing of information on standard medical databases or other similar applications. Preferably, communication module 800 is connected to other modules via USB, BioBus, or other communication protocol that allows communication of signals/data among the modules. The communication module 800 may be capable of transmitting exposure, safety and system information via a variety of wireless communication protocols, as mentioned above, for the analysis of a patient or test subject by medical professionals at a remote location.

### Methods

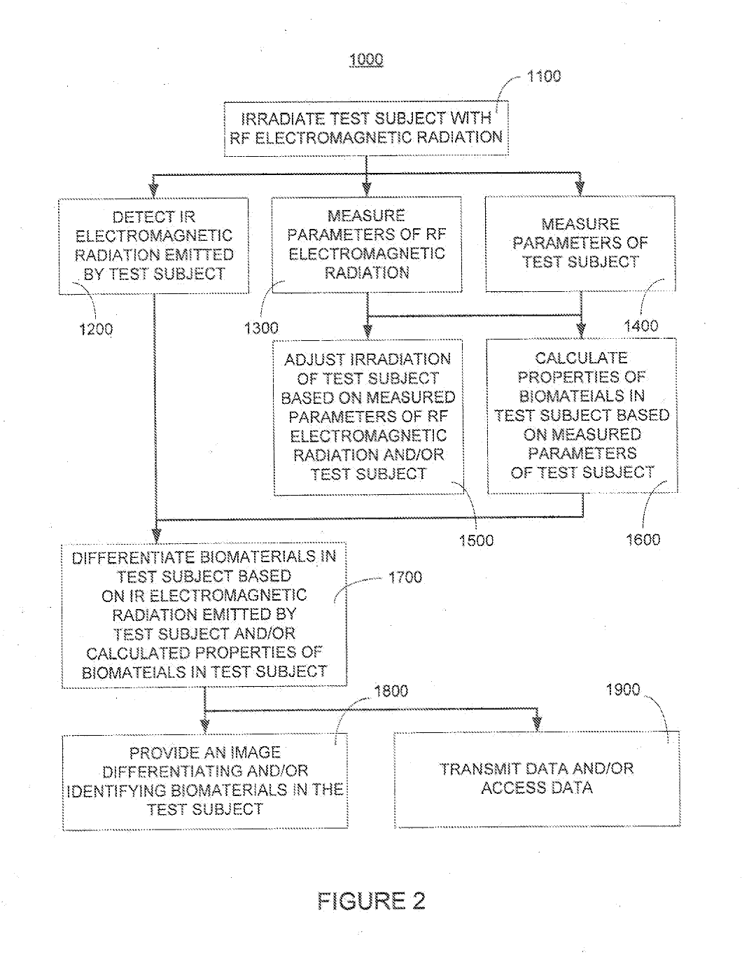

In accordance with another aspect of the invention, provided are methods for multispectral scanning and detection of biomaterials in nanos utilized in the treatment of the test subject or patient 20. FIG. 2 shows a flowchart of one exemplary implementation of a method 1000 in accordance with the present invention. It will be apparent to those skilled the art that the steps shown in FIG. 2 may be performed in a different order. Further, the steps show in FIG. 2 may be performed simultaneously, sequentially or separately. Still further, some of the steps shown in FIG. 2 may be omitted and/or additional steps (not shown) may be included.

In one implementation, method 1000 begins with step 1100 by irradiating the nanos with RF electromagnetic radiation. More particularly, step 1100 may comprise irradiating nanos with electromagnetic radiation, preferably in the 1 GHz to 3 GHz frequency range. Nanos may be irradiated with RF electromagnetic energy, for example, by operation of scanning module 100 as described above with reference to FIG. 1.

In another implementation, at step 1200 is detecting IR electromagnetic radiation emitted by nanos as it absorbs RF electromagnetic energy and converts it into thermal energy. Step 1200 may be performed, for example, by operation of detection module 200 as described above with reference to FIG. 1.

In another implementation, at step 1300 is measuring and/or calculating parameters of the RF electromagnetic radiation impinged on nanos. In particular, step 1300 may comprise performing calculations to determine different aspects of the system's 10 performance, such as EIRP, power density, path loss, power incident, and power reflected. Further, step 1300 may comprise performing calculations to determine whether the electromagnetic radiation complies with FCC MPE limits for power density. Step 1300 may be performed, for example, by operation of scanning module 100 and processing module 300 as described above with reference to FIG. 1.

In another implementation, at step 1400 is measuring and/or calculating parameters of nanos during irradiation. In particular step 1400 may comprise measuring and/or calculating electromagnetic energy absorbed by nanos, electromagnetic energy reflected by nanos, depth of penetration of electromagnetic energy into nanos, initial temperature of nanos, and final temperature of nanos. Step 1400 may be performed, for example, by operation of detection module 200 and processing module 300 as described above with reference to FIG. 1.

At step 1500 is adjusting irradiation of nanos based on measured and/or calculated parameters of the RF electromagnetic radiation and nanos to control the electromagnetic radiation output to comply with FCC MPE limits while maximizing the depth of penetration to ensure proper scanning of nanos as described above. In particular, step 1500 may comprise adjusting the output power, antenna gain, and frequency of a signal generator to obtain maximum penetration of nanos while complying with FCC MPE limits. Step 1500 may be performed, for example, by operation of processing module 300, control module 400, and scanning module 100 as described above with reference to FIG. 1.

The control module can be used to adjust the irradiation of the nanos so that they generate thermal energy which can be applied to treat the patient.

At step 1600 is calculating electromagnetic properties of biomaterials in nanos based on measured and/or calculated parameters of nanos during irradiation. More particularly, step 1600 may comprise calculating electromagnetic properties of the biomaterials in nanos, such as relative static permittivity (ε), magnetic permeability (μ), and thermal conductivity (κ), based on measured and calculated parameters of nanos, such as attenuation α of electromagnetic radiation, absorption/reflection of electromagnetic radiation, depth of penetration electromagnetic radiation, emission of IR electromagnetic radiation, and change in temperature. Step 1600 may be performed, for example, by operation of processing module 300 and detection module 200 as described above with reference to FIG. 1.

The next step 1700 is differentiating and/or identifying biomaterials in nanos based on IR electromagnetic radiation emitted by different biomaterials and/or the calculated electromagnetic properties of different biomaterials. Step 1700 may be performed, for example, by operation of detection module 200, processing module 300, and data module 500 as described above with reference to FIG. 1.

The next step 1800 is providing an image of a scanned portion of nanos differentiating and/or identifying different biomaterials. Step 1800 may be performed, for example, by operation of data module 500, an optional imaging module 600, and a display module 700 as described above with reference to FIG. 1.

The next step 1900 is transmitting data to a medical practitioner and/or accessing data from a medical database for the purpose of diagnosing nanos. Step 1900 may be performed, for example, by operation of communication module 800 via a wireless air interface such as Bluetooth, WiFi, WiMax or the like.

The entire system described above may be contained in a portable and robust package. The system may also include a small LCD display so that the system may be easily deployed in field applications. Further, the system may be packaged in a unit of approximately the same weight and power requirements as a standard laptop or notebook computer and may be powered by a power supply similar to that used for such computer platforms.

We claim:

1. A method for scanning and detecting biomaterials in nanoparticles utilized in the treatment of the test subject or patient for medical diagnosis, which comprises:
irradiating with non-ionizing radio frequency (RF) electromagnetic radiation at least a portion of the nanoparticles utilized in the treatment of the test subject or patient, with the radiation having a frequency of 1 to 40 GHz and at a density that is between 1 and 100 mW/cm² and at a time weighted average power density that would not harm or injure the subject;
delivering the irradiated nanoparticles to the test subject or patient; and
detecting infrared (IR) electromagnetic radiation emitted by the irradiated nanoparticles to assist in determining how to proceed with the subsequent treatment of the test subject or patient.

2. The method of claim 1 which further comprises providing data from the nanoparticles utilized in the subsequent treatment of the test subject or patient to evaluate a dosage of biomaterials.

3. The method of claim 1 which further comprises measuring parameters of the irradiation of the nanoparticles utilized in the subsequent treatment of the test subject or patient.

4. The method of claim 3 which further comprises adjusting the irradiation of the nanoparticles utilized in the subsequent treatment of the test subject or patient based on the measured parameters of the irradiation.

5. The method of claim 1 which further comprises measuring parameters of the nanoparticles utilized in the subsequent treatment of the test subject or patient during irradiation.

6. The method of claim 5 which further comprises determining electromagnetic properties of different nanoparticles utilized in the subsequent treatment of the test subject or patient based on the measured parameters of the nanoparticles.

7. The method of claim 5 which further comprises differentiating nanoparticles utilized in the subsequent treatment of the test subject or patient based on the electromagnetic properties of different biomaterials.

8. The method of claim 1 which further comprises transmitting or accessing data for diagnosing the nanoparticles utilized in the subsequent treatment of the test subject or patient.

9. The method of claim 1 which further comprises presenting a thermal image of the irradiated nanoparticles utilized in the subsequent treatment of the test subject or patient by differentiating different levels of IR electromagnetic radiation emitted.

10. The method of claim 1 which further comprises sensing distance of travel of the electromagnetic irradiation of the nanoparticles to determine whether the electromagnetic radiation requires adjustment.

11. The method of claim 8 wherein the data is transmitted to remote locations for further analysis.

12. The method of claim 1 which further comprises displaying data relating to irradiated nanoparticles utilized in the subsequent treatment of the test subject or patient.

13. The method of claim 12 which further comprises differentiating the displayed data to identify nanoparticles corresponding to different emissions of IR electromagnetic radiation.

14. The method of claim 1 wherein the irradiation has a frequency of 1 GHz to 3 GHz.

15. The method of claim 3 wherein the measuring of the parameters of the irradiated nanoparticles includes performing calculations to determine system performance relating to incident RF electromagnetic radiation, effective isotropic irradiated power, power density, path loss, incident power, and reflected power.

16. The method of claim 3 wherein the RF electromagnetic radiation applied to the nanoparticles is adjusted to obtain a maximized depth of penetration to ensure proper scanning of the test subject.

17. The method of claim 16 wherein the differentiating of the biomaterials is based on measuring or calculating attenuation of electromagnetic radiation, absorption or reflection of electromagnetically irradiated nanoparticles, depth of electromagnetic radiation penetration, emission of RF electromagnetic radiation, or created thermal energy.

18. The method of claim 1 wherein the irradiation is applied to the nanoparticles when they are present on or within the test subject or patient.

19. The method of claim 1 wherein the irradiation is applied to the nanoparticles when they are present within the test subject or patient and the radiation is applied to maximize depth of penetration to ensure proper scanning of the nanoparticles.
